(19) United States
(12) Patent Application Publication
(10) Pub. No.: US 2026/0248370 A1
(43) Pub. Date: Aug. 27, 2026

(54) END CAP HAVING ONE OR MORE ELECTRODES, RELATED SYSTEMS, AND RELATED METHODS OF USE

(71) Applicant: Boston Scientific Scimed, Inc., Maple Grove, MN (US)

(72) Inventors: Charles R. PARAISO, Castle Rock, CO (US); Barry D. WEITZNER, Great Barrington, MA (US)

(73) Assignee: Boston Scientific Scimed, Inc., Maple Grove, MN (US)

(21) Appl. No.: 19/546,631

(22) Filed: Feb. 23, 2026

Related U.S. Application Data
(60) Provisional application No. 63/764,040, filed on Feb. 27, 2025.

(51) Int. Cl. A61B 1/00; A61B 1/018; A61B 18/12; A61B 18/14; A61B 90/00; A61B 18/00

(52) U.S. Cl. CPC A61B 1/00137; A61B 1/0008; A61B 1/00114; A61B 1/018; A61B 18/1206; A61B 18/14; A61B 90/08; A61B 2018/00589; A61B 2018/00601; A61B 2018/00607; A61B 2018/00982; A61B 2018/1253; A61B 2018/126; A61B 2018/1405; A61B 2090/0807

(57) ABSTRACT

Medical systems are described, including a medical system including a medical device and an end cap. The medical device includes a handle and a shaft extending distally from the handle to a distal end. The shaft includes a working channel. The end cap is coupled to the distal end of the shaft and includes a first portion, a second portion, and a protrusion. The first portion has a lumen and the distal end of the shaft is positioned within the lumen. The second portion extends distally from the first portion and includes a wall defining a chamber. The chamber is in communication with the working channel of the shaft. The protrusion extends distally from the second portion and a distal end of the protrusion includes an electrode.

### CROSS-REFERENCE TO RELATED APPLICATIONS

This application claims the benefit of priority to U.S. Provisional Application No. 63/764,040, filed on Feb. 27, 2025, which is incorporated by reference herein in its entirety.

### TECHNICAL FIELD

Various aspects of this disclosure relate generally to end caps for medical devices, related systems, and related methods of use. In particular, aspects of this disclosure relate to end caps having one or more electrodes, e.g., for cutting tissue and/or coagulating tissue.

### BACKGROUND

Medical procedures may involve removing a lesion or other tissue from a body lumen. For example, endoscopic mucosal resection (EMR) and endoscopic submucosal dissection (ESD) utilize multiple medical instruments to remove cancerous lesions or other tissue from the gastrointestinal tract. For example, medical instruments (e.g., an electrosurgical knife or a hemostatic device) may be passed through a working channel of an endoscope in order to cut a lesion or to stop bleeding of tissue. The endoscope and the medical instruments may be moved or controlled independent of one another, which may present challenges during a medical procedure. For example, inadvertent movement of the endoscope and/or medical instruments during the medical procedure may increase the duration of the procedure, decrease the efficiency of the procedure, or otherwise negatively affect the procedure. Furthermore, during the procedure, portions of the cut lesion or other tissue may interfere with the visualization and/or treatment of the lesion or other tissue. In addition, an endoscope that includes a single working channel may not be suitable for medical procedures involving removal or treatment of a lesion or other tissue. For example, an operator may require a first medical instrument to grasp a lesion or other tissue and a second medical instrument to cut the lesion or other tissue, which may require multiple working channels or multiple devices to be inserted into the body lumen.

### SUMMARY

Each of the aspects disclosed herein may include one or more aspects of the features described in connection with any of the other disclosed aspects.

In an aspect, a medical system may include a medical device and an end cap. The medical device may include a handle and a shaft extending distally from the handle to a distal end. The shaft may include a working channel. The end cap may be coupled to the distal end of the shaft and may include a first portion, a second portion, and a protrusion. The first portion may have a lumen, and the distal end of the shaft may be positioned within the lumen. The second portion may extend distally from the first portion and may include a wall defining a chamber. The chamber may be in communication with the working channel of the shaft. The protrusion may extend distally from the second portion, and a distal end of the protrusion may include an electrode.

Any of the devices, systems, or methods disclosed herein may include any of the following features, alone or in any combination. The electrode may be configured to be energized to cut tissue. The electrode may be a first electrode, and the protrusion may further include a second electrode. The second electrode may be configured to be energized to coagulate tissue. A width of the protrusion may be less than a width of the first portion of the end cap. The width of the protrusion may be less than a width of at least proximal portions of the second portion of the end cap. The protrusion may include a first surface and a second surface, and the first surface may be on an opposite side of the protrusion from the second surface. The electrode may be disposed on a portion of the first surface at the distal end of the protrusion or on a portion of the second surface at the distal end of the protrusion. The second electrode may be disposed on a portion of the second surface. The electrode may be disposed on a distal edge of the protrusion and the distal edge may be curved. The electrode may be monopolar or bipolar. One or more wires may extend proximally from the electrode, through the first portion and the second portion, and external to the shaft to an energy source. The wall of the second portion may extend less than 360° around a central longitudinal axis of the end cap. The wall of the second portion may include an edge that extends distally at an angle relative to the central longitudinal axis of the end cap. The end cap may include a marker positioned on an outer surface of the end cap opposite the protrusion. The protrusion of the end cap and a distal opening of the working channel of the shaft may be on opposite sides of the central longitudinal axis of the end cap.

In another aspect, an end cap for a medical device may include a first portion, a second portion, and a protrusion. The first portion may include a lumen configured to receive a distal end of a shaft of the medical device. The second portion may extend distally from the first portion. The second portion may include a wall defining a chamber. The protrusion may extend distally from the second portion. The protrusion may include a first surface, a second surface, and one or more electrodes configured to be energized to deliver energy to tissue. The first surface may be on an opposite side of the protrusion from the second surface. Each of the first surface and the second surface may be planar.

Any of the devices, systems, and methods disclosed herein may include any of the following features, alone or in any combination. The distal end of the protrusion may be curved. The distal end of the protrusion may include the one or more electrodes. The one or more electrodes may include a first electrode and a second electrode. The first electrode may be disposed on the first surface. The second electrode may be disposed on the second surface.

A further aspect relates to a method of treating tissue. The method may include navigating a medical system through a body lumen to a target tissue, where the medical system may include a medical device and an end cap. The medical device may include a shaft having a working channel. The end cap may be coupled to a distal end of the shaft. The end cap may include a protrusion having an electrode at a distal end of the protrusion. At least a distal portion of the protrusion may be flat. The method may include extending a medical instrument through the working channel of the shaft, grasping a portion of the target tissue with the medical instrument to apply traction to the target tissue, energizing the electrode, and manipulating one or more of the shaft of the medical device or the medical instrument such that at least a portion of the electrode contacts a portion of the target tissue to cut the target tissue. The protrusion may include a distal edge. The distal edge may be curved.

### BRIEF DESCRIPTION OF FIGURES

The accompanying drawings, which are incorporated in and constitute a part of this application, illustrate exemplary aspects of this disclosure and together with the description, serve to explain the principles of this disclosure.

FIGS. 1A and 1B illustrate an exemplary medical system including a medical device and an end cap coupled to a distal end of the medical device, according to aspects of this disclosure.

FIGS. 2A-2D illustrate various views of the end cap of FIGS. 1A and 1B, according to aspects of this disclosure.

FIG. 3 illustrates an exemplary usage of the medical system of FIGS. 1A and 1B at a target site, according to aspects of this disclosure.

### DETAILED DESCRIPTION

Particular aspects of this disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although endoscopes are referenced herein for illustration purposes, it will be appreciated that this disclosure encompasses any suitable delivery device or medical device configured to allow an operator to access and view internal body anatomy of a subject (e.g., patient) and/or to deliver medical instruments, such as, for example, biopsy forceps, graspers, baskets, snares, probes, scissors, retrieval devices, lasers, and other tools, into the subject’s body. The medical devices herein may be inserted into a variety of body lumens and/or cavities, such as, for example, the urinary tract or gastrointestinal tract. It will be appreciated that, unless otherwise specified, bronchoscopes, duodenoscopes, ureteroscopes, gastroscopes, endoscopic ultrasonography (“EUS”) scopes, colonoscopes, laparoscopes, cystoscopes, aspiration scopes, sheaths, catheters, or any other suitable delivery device or medical device may be used in connection with the features described herein.

This disclosure includes end caps for medical devices. Features of the end caps herein may improve control of components involved with cutting tissue and/or stopping bleeding of tissue (e.g., tissue coagulation) by coupling movement of the components with movement of a medical device (e.g., 1-to-1 movement), among other aspects. Improved control of tissue cutting and/or tissue coagulation components may reduce procedure times and may improve efficiency and safety of medical procedures, such as EMR or ESD, among other aspects. The end caps herein may include tissue cutting and/or tissue coagulation components, which may help reduce the need for multiple working channels in a medical device (or multiple medical devices) to deliver multiple medical instruments to a target site.

According to some aspects of this disclosure, the end cap may include one or more electrodes. The end cap may function as a monopolar device or a bipolar device. The end cap may include a protrusion, and the electrode(s) may be disposed on or integrated with surfaces and/or edges of the protrusion. The electrode(s) may be used to cut tissue and/or coagulate tissue during a medical procedure. According to some aspects of this disclosure, the protrusion of the end cap may be used to move or manipulate tissue during a medical procedure. For example, the protrusion of the end cap may be used for blunt dissection.

According to some aspects of this disclosure, the end cap may be coupled to a distal end of a medical device, while allowing other medical instruments (e.g., an injection needle, a grasper, forceps, a snare, etc.) to be extended through a working channel of the medical device. For example, during a medical procedure, an operator may grasp tissue using a medical instrument (e.g., a grasper) extending through the working channel of the medical device and may cut the tissue using the protrusion of the end cap at the same time. The end cap may be rigidly coupled to the distal end of the medical device, which may allow for an operator to control movement of the protrusion of the end cap via movement of the medical device (e.g., 1-to-1 movement). For example, the distal end of the medical device may be moved (e.g., moved in a longitudinal direction, deflected, or rotated) to correspondingly move the protrusion of the end cap.

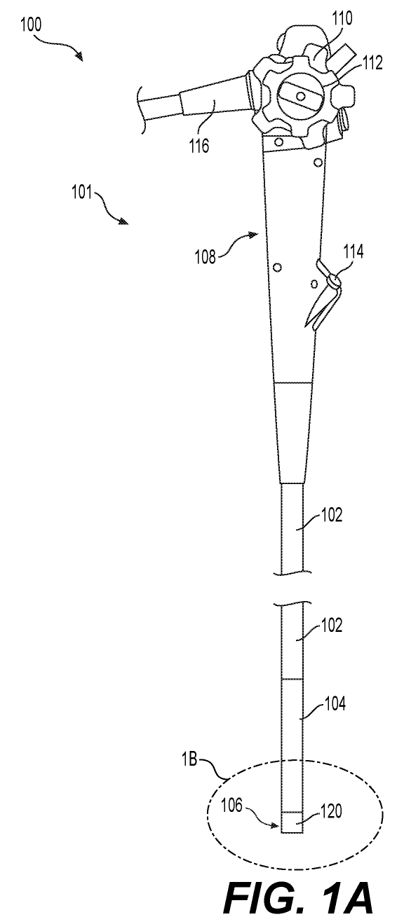

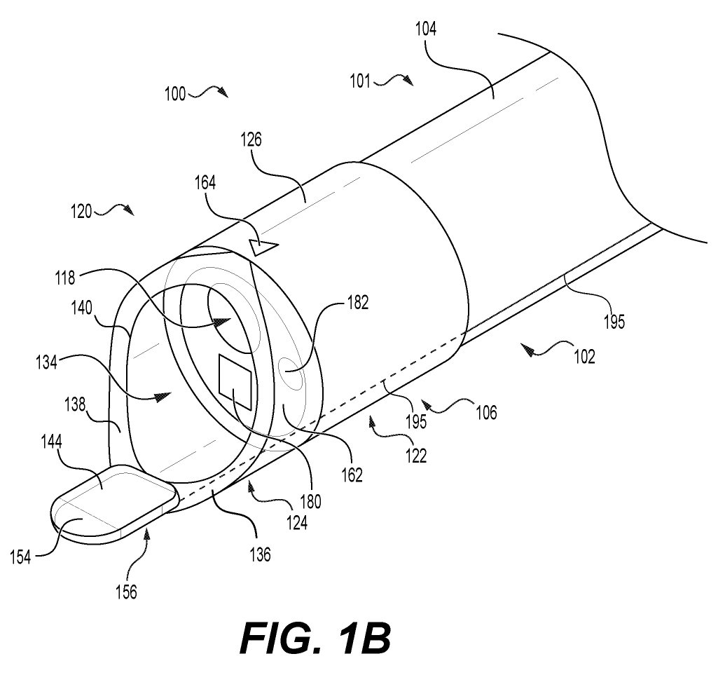

FIG. 1A illustrates an exemplary medical system 100, and FIG. 1B illustrates a distal portion of medical system 100. Medical system 100 may include a medical device 101 (e.g., an endoscope) and an end cap 120 coupled to a distal portion of medical device 101.

Medical device 101 may include a handle 108 and a shaft 102, with shaft 102 extending distally from handle 108. Shaft 102 may include an articulation section 104 and a distal tip or end 106. As shown in FIG. 1B, a distalmost face 162 of shaft 102 may include an imaging device 180 (e.g., a camera, an imager, etc.), one or more light sources 182 (e.g., light emitting diode(s), optical fibers, etc.), and a distal opening in communication with a working channel 118 extending through shaft 102. Referring back to FIG. 1A, handle 108 may include a port 114 in fluid communication with working channel 118. Handle 108 may include one or more actuators, e.g., a first actuator 110 and a second actuator 112 (e.g., knobs or levers), for controlling articulation section 104 of shaft 102. For example, actuators 110, 112 may control movement of articulation section 104 of shaft 102 in multiple directions, e.g., movement along different planes. Handle 108 may be coupled to an umbilicus 116. Umbilicus 116 may extend from handle 108 to capital equipment, such as electronic controller(s), and/or source(s) of air, water, and/or suction.

End cap 120 may be secured to distal end 106 of shaft 102 via a friction fit or other coupling mechanism. As shown in FIG. 1B, end cap 120 may include a cylindrical or tubular first portion 122 (e.g., a proximal portion) and a tapering second portion 124 (e.g., a distal portion). Distal end 106 of shaft 102 may be received or otherwise positioned within first portion 122 of end cap 120. For example, distal end 106 of shaft 102 may be received or otherwise positioned within a cylindrical body 126 of first portion 122. Second portion 124 of end cap 120 may extend distal to distalmost face 162 of shaft 102. Second portion 124 of end cap 120 may include a wall 136 defining an open chamber 134 that is in communication with working channel 118 of shaft 102. Wall 136 and/or one or more other portions of end cap 120 may be formed of a transparent material, which may assist with preventing wall 136 or other portions of end cap 120 from interfering with illumination/visualization of a target site during a procedure. Chamber 134 may include a distal opening 140 defined by an edge 138 of wall 136. Chamber 134 may allow for an operator to visualize a target site and/or extend medical instruments distally from working channel 118 of shaft 102 to a target site, for example.

As discussed in detail below, in some aspects, end cap 120 includes a protrusion 144 extending distally from second portion 124. Protrusion 144 includes one or more electrodes, e.g., a first electrode 154 and a second electrode 156. Electrodes 154, 156 may be coupled to an energy source (e.g., an electrosurgical unit) to deliver current to tissue. For example, first electrode 154 may be used to cut tissue (e.g., a cutting electrode), and second electrode 156 may be used to coagulate tissue (e.g., a coagulation electrode). Electrodes 154, 156 may be coupled to the energy source via one or more wires 195. For example, the wire(s) may extend from electrodes 154, 156 to the energy source. Wire(s) 195 may extend through portions of end cap 120. As shown in FIG. 1B, wire(s) 195 may extend proximally from electrodes 154, 156, through portions of wall 136 of second portion 124, through walls of cylindrical body 126 of first portion 122, and external to or outside of shaft 102 to the energy source. For example, a proximal end of end cap 120 may include one or more openings in communication with one or more lumens of end cap 120. In some aspects, although not shown, medical system 100 may include an outer sheath, which may radially surround shaft 102 and wire(s) 195. Wire(s) 195 may extend through the opening(s) and the lumen(s) of end cap 120. In an alternative, wire(s) 195 may extend through working channel 118. In another alternative, wire(s) 195 may be built into walls of shaft 102 and may include one or more electrical contacts that couple with one or more electrical contacts of end cap 120 when end cap 120 is coupled to shaft 102.

Electrodes 154, 156 may be monopolar or bipolar. In at least one example in which electrodes 154, 156 are bipolar, protrusion 144 may further include one or more return electrodes. In an alternative, second electrode 156 may be a return electrode for first electrode 154. In another alternative, second electrode 156 may be omitted.

End cap 120 may include one or more markers 164 (e.g., positioning markers) positioned on an outer surface of end cap 120. Marker(s) 164 may be positioned on a side of end cap 120 opposite protrusion 144 relative to a central longitudinal axis of end cap 120. In these aspects, marker(s) 164 may be at least partially aligned with the distal opening of working channel 118 when end cap 120 is coupled to shaft 102. Marker(s) 164 may assist an operator with orienting end cap 120 on shaft 102, such that protrusion 144 is opposite the distal opening of working channel 118. Having protrusion 144 opposite working channel 118 may assist with preventing protrusion 144 from obstructing a field of view and/or interfering with movement of medical instruments that extend through working channel 118 during a medical procedure, for example.

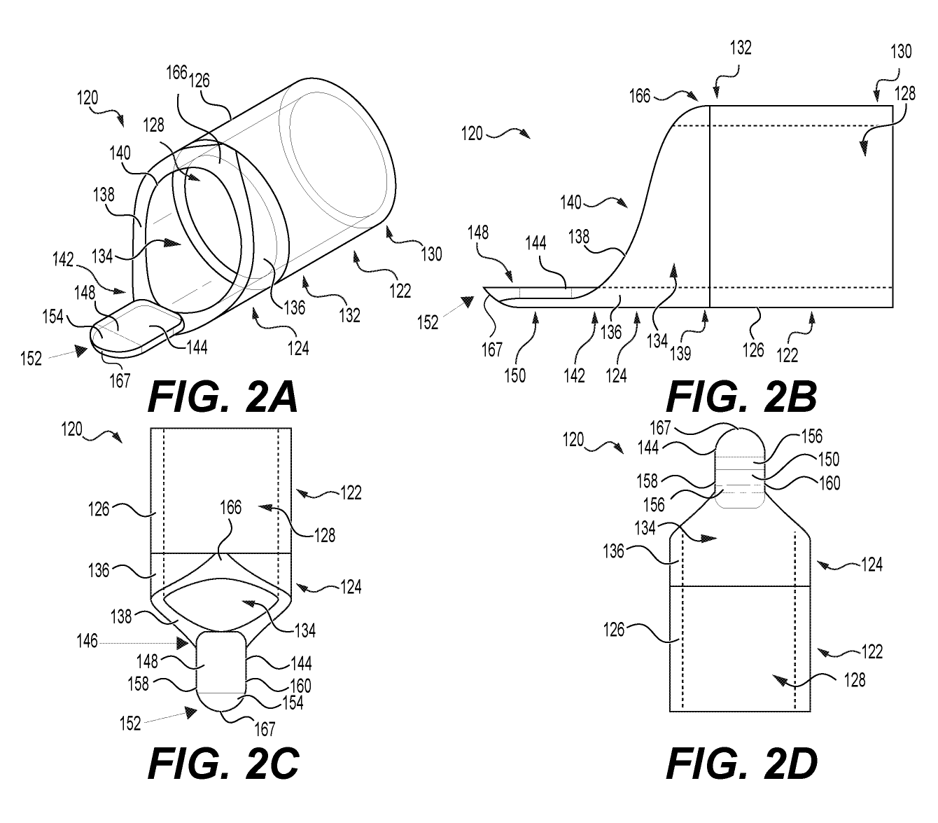

End cap 120 will now be further described with reference to FIGS. 2A-2D. FIG. 2A illustrates a perspective view of end cap 120, FIG. 2B illustrates a side view of end cap 120, FIG. 2C illustrates a top view of end cap 120, and FIG. 2D illustrates a bottom view of end cap 120.

As mentioned above, end cap 120 may include first portion 122 and second portion 124. First portion 122 may be integrally formed with second portion 124. In alternatives, first portion 122 may be formed as a separate piece and coupled to second portion 124. Cylindrical body 126 of first portion 122 may extend from a proximal end 130 to a distal end 132. Walls of cylindrical body 126 may define a lumen 128 of first portion 122. Lumen 128 of first portion 122 is in communication with chamber 134 of second portion 124. Lumen 128 of first portion 122 may be sized and shaped to receive distal end 106 of shaft 102. A length of cylindrical body 126 (e.g., in a direction parallel to a longitudinal axis of end cap 120) may range from about 5 mm to about 30 mm, such as from about 10 mm to about 20 mm. In some aspects, the length of cylindrical body 126 is about 10 mm.

Portions of wall 136 may extend less than 360° around a central longitudinal axis of end cap 120, which may assist with improved visualization of a target site, for example. As shown in FIG. 2B, edge 138 of wall 136 may extend distally at an angle relative to the central longitudinal axis of end cap 120. Edge 138 may include a proximal end 166 that is proximate or adjacent to distal end 132 of first portion 122. Edge 138 of wall 136 may taper from proximal end 166 of edge 138 to distal end 142 of second portion 124. For example, distal opening 140 of chamber 134 may taper in a distal direction. A length from a proximal end 139 of wall 136 to distal end 142 of wall 136 (e.g., in a direction parallel to the longitudinal axis of end cap 120) may range from about 4 mm to about 15 mm, such as from about 6 mm to about 9 mm. In some aspects, the length from proximal end 139 of wall 136 to distal end 142 of wall 136 is about 7.5 mm. A radius of distal opening 140 of chamber 134 may range from about 3 mm to about 10 mm, such as from about 5 mm to about 8 mm. In some aspects, the radius of distal opening 140 of chamber 134 is about 5.5 mm. A thickness of wall 136 (e.g., in a direction substantially perpendicular to the longitudinal axis of end cap 120) may range from about 0.5 mm to about 4 mm, such as from about 1 mm to about 2 mm. In some aspects, the thickness of wall 136 is about 1 mm.

Protrusion 144 may extend distally from distal end 142 of second portion 124. In some examples, protrusion 144 may be integrally formed with second portion 124. In alternatives, protrusion 144 may be formed as a separate piece and coupled to second portion 124. In some examples, protrusion 144 may be approximately as rigid or as flexible as remaining portions of end cap 120. In other examples, protrusion 144 may be more flexible or more rigid than remaining portions of end cap 120. Protrusion 144 may extend from a proximal end 146 (FIG. 2C) to a rounded or curved distal end 152. Protrusion 144 may be substantially flat (e.g., including substantially planar surfaces). In some aspects, at least a distal portion of protrusion 144 may be flat. Protrusion 144 may include a first surface 148 and a second surface 150 (e.g., as shown in FIG. 2D) extending from proximal end 146 to distal end 152. Surfaces 148, 150 may each be a planar surface. First surface 148 may be on an opposite side of protrusion 144 from second surface 150. First surface 148 may face a direction towards (e.g., radially toward) the central longitudinal axis of end cap 120, and second surface 150 may face a direction away (e.g., radially outward) from the central longitudinal axis of end cap 120. For example, first surface 148 may be a top, planar surface and second surface 150 may be a bottom, planar surface.

Protrusion 144 may include a first side edge 158, a second side edge 160, and a distal edge 167. Edges 158, 160, 167 may each extend between first surface 148 and second surface 150. As shown in FIGS. 2C and 2D, each side edge 158, 160 may extend substantially parallel to the longitudinal axis of end cap 120, and distal edge 167 may curve outward between side edges 158, 160. As shown in FIG. 2B, distal edge 167 may be beveled such that a distalmost end of first surface 148 may be distal to a distalmost end of second surface 150. In some examples, one or more of edges 158, 160, 167 may be a rounded edge.

Protrusion 144 may have a narrower profile as compared to remaining portions of end cap 120. A width of protrusion 144 (e.g., in a direction perpendicular to the longitudinal axis of end cap 120) may be less than a width of first portion 122 of end cap 120. The width of protrusion 144 may be less than a width of at least proximal portions of second portion 124 of end cap 120. For example, the width of protrusion 144 may range from about 2 mm to about 10 mm, such as from about 3 mm to about 5 mm. In some aspects, the width of protrusion 144 is about 4 mm. A length of protrusion 144 (e.g., in a direction parallel to the longitudinal axis of end cap 120) may range from about 4 mm to about 15 mm, such as from about 5 mm to about 7 mm. In some aspects, the length of protrusion 144 is about 6 mm. A radius of curvature of distal end 152 of protrusion 144 may range from about 0.5 mm to about 6 mm, such as from about 1 mm to about 3 mm. In some aspects, the radius of curvature of distal end 152 of protrusion 144 is about 2 mm.

First electrode 154 (e.g., the cutting electrode) may be positioned at distal end 152 of protrusion 144. First electrode 154 may be disposed on or integrated with portions of first surface 148 at distal end 152 of protrusion 144, disposed on or integrated with portions of second surface 150 at distal end 152 of protrusion 144, and/or disposed on or integrated with distal edge 167 of protrusion 144. As shown in FIG. 2D, second electrode 156 (e.g., the coagulation electrode) may be disposed on or integrated with portions of second surface 150 of protrusion 144. Second electrode 156 may be positioned proximal to distal end 152 of protrusion 144 and may extend from first side edge 158 to second side edge 160.

In some aspects, protrusion 144 may include second electrode 156 and one or more additional second electrodes 156 (shown in dashed lines in FIG. 2D) disposed on or integrated with portions of second surface 150 of protrusion 144. In these aspects, a first set of or one or more second electrodes 156 may have a first polarity, and a second set of or one or more other second electrodes 156 may have a second polarity. Electrodes 156 may be positioned on second surface 150 of protrusion 144 parallel to one another or otherwise spaced apart. For example, second surface 150 of protrusion 144 may include alternating first polarity and second polarity electrodes 156 from proximal end 146 to distal end 152 of protrusion 144. This may allow an operator to selectively deliver energy to a target site. In some aspects, the energy delivered to the target site via second electrode(s) 156 may be different from the energy delivered to the target site via first electrode 154. For example, in some aspects, the energy delivered to the target site via second electrode(s) 156 may be used to help with hemostasis of one or more portions of the target site (e.g., to coagulate and/or cauterize desired portions of tissue).

The narrow profile of protrusion 144 and the positioning of electrodes 154, 156 on protrusion 144 (e.g., positioning of first electrode 154 at distal end 152 of protrusion 144) may assist an operator with cutting underneath lifted tissue, cutting small areas of tissue, and/or otherwise assist with delivering energy more accurately during a procedure.

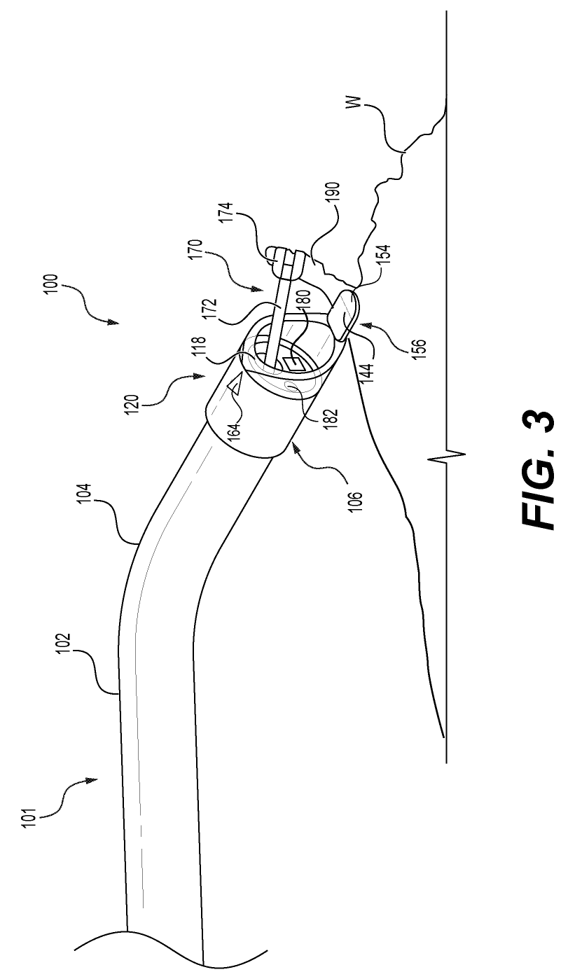

A method of using medical system 100 to treat tissue will now be described with reference to FIG. 3, which illustrates a distal portion of medical system 100, including medical device 101 and end cap 120, at a target site.

An operator may secure end cap 120 to distal end 106 of shaft 102. The operator may use marker(s) 164 to orient protrusion 144 opposite the distal opening of working channel 118 (e.g., relative to the central longitudinal axis of shaft 102). The operator may navigate medical system 100 through a body lumen (e.g., the gastrointestinal tract) of a subject and to target tissue 190. Once distal end 106 of shaft 102 and end cap 120 are proximate target tissue 190, the operator may extend a medical instrument 170 distally through or otherwise distally out of working channel 118. Medical instrument 170 may include a shaft 172 and an end effector 174 (e.g., graspers, forceps, etc.). The operator may use end effector 174 to grasp and apply traction to target tissue 190. End effector 174 may pull target tissue 190 away from surrounding tissue wall W of the body lumen. The traction applied to target tissue 190 may facilitate cutting or otherwise treatment of target tissue 190. Although not shown, in some aspects, one or more electrodes (e.g., first electrode 154) may be energized and applied to tissue wall W, for example, to initially cut target tissue 190.

As shown in FIG. 3, while the traction applied to target tissue 190 is maintained using medical instrument 170, the operator may activate an energy source to supply energy (e.g., current) to first electrode 154 to cut target tissue 190 from tissue wall W. Medical instrument 170 may be positioned opposite protrusion 144, which may assist the operator with pulling or lifting target tissue 190 away from tissue wall W, while allowing access for protrusion 144 to cut underneath the lifted target tissue 190.

The operator may manipulate and/or move protrusion 144 to cut target tissue 190 by manipulating and/or moving shaft 102. For example, the operator may manipulate and/or move shaft 102 to position first electrode 154 of protrusion 144 proximate target tissue 190 or to position electrode 154 of protrusion 144 in contact with a portion of target tissue 190. For example, during the procedure, the operator may move shaft 102 proximally or distally using handle 108 to move protrusion 144 proximally or distally, articulate articulation section 104 of shaft 102 using actuators 110, 112 of handle 108 to deflect protrusion 144, and/or rotate shaft 102 using handle 108 to rotate protrusion 144. Imaging device 180 and one or more light sources 182 at distal end 106 of shaft 102 may assist with visualizing medical instrument 170 and protrusion 144 throughout the procedure.

In some examples, if bleeding at tissue wall W occurs, the operator may energize second electrode 156 and apply second electrode 156 (e.g., the coagulation electrode) to a portion of tissue wall W to stop bleeding at tissue wall W.

It will be appreciated that throughout a procedure, the operator may supply current to electrodes 154, 156 simultaneously or at different times. For example, the operator may activate (e.g., energize) or deactivate electrodes 154, 156 depending on whether cutting of tissue and/or coagulation is needed. In some examples, protrusion 144 may be used to move, manipulate, or otherwise separate layers of tissue during a procedure, e.g., when electrodes 154, 156 are deactivated (e.g., not energized).

It will be apparent to those skilled in the art at various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A medical system comprising:
a medical device comprising:
a handle; and
a shaft extending distally from the handle to a distal end, wherein the shaft includes a working channel; and
an end cap coupled to the distal end of the shaft, the end cap comprising:
a first portion having a lumen, wherein the distal end of the shaft is positioned within the lumen;
a second portion extending distally from the first portion, wherein the second portion includes a wall defining a chamber, and wherein the chamber is in communication with the working channel of the shaft; and
a protrusion extending distally from the second portion, wherein a distal end of the protrusion includes an electrode.

2. The medical system of claim 1, wherein the electrode is configured to be energized to cut tissue.

3. The medical system of claim 2, wherein the electrode is a first electrode, and wherein the protrusion further comprises a second electrode.

4. The medical system of claim 3, wherein the second electrode is configured to be energized to coagulate tissue.

5. The medical system of claim 1, wherein a width of the protrusion is less than a width of the first portion of the end cap, and wherein the width of the protrusion is less than a width of at least proximal portions of the second portion of the end cap.

6. The medical system of claim 3, wherein the protrusion includes a first surface and a second surface, and wherein the first surface is on an opposite side of the protrusion from the second surface.

7. The medical system of claim 6, wherein the electrode is disposed on a portion of the first surface at the distal end of the protrusion or on a portion of the second surface at the distal end of the protrusion.

8. The medical system of claim 6, wherein the second electrode is disposed on a portion of the second surface.

9. The medical system of claim 1, wherein the electrode is disposed on a distal edge of the protrusion, and wherein the distal edge is curved.

10. The medical system of claim 1, wherein the electrode is monopolar or bipolar.

11. The medical system of claim 1, wherein one or more wires extend proximally from the electrode, through the first portion and the second portion, and external to the shaft to an energy source.

12. The medical system of claim 1, wherein the wall of the second portion extends less than 360° around a central longitudinal axis of the end cap.

13. The medical system of claim 1, wherein the wall of the second portion includes an edge that extends distally at an angle relative to a central longitudinal axis of the end cap.

14. The medical system of claim 1, wherein the end cap includes a marker positioned on an outer surface of the end cap opposite the protrusion.

15. The medical system of claim 1, wherein the protrusion of the end cap and a distal opening of the working channel of the shaft are on opposite sides of a central longitudinal axis of the end cap.

16. An end cap for a medical device, the end cap comprising:
a first portion including a lumen, wherein the lumen is configured to receive a distal end of a shaft of the medical device;
a second portion extending distally from the first portion, wherein the second portion includes a wall defining a chamber; and
a protrusion extending distally from the second portion, the protrusion comprising:
a first surface;
a second surface, wherein the first surface is on an opposite side of the protrusion from the second surface, and wherein each of the first surface and the second surface is planar; and
one or more electrodes configured to be energized to deliver energy to tissue.

17. The end cap of claim 16, wherein the distal end of the protrusion is curved, and wherein the distal end of the protrusion includes the one or more electrodes.

18. The end cap of claim 16, wherein the one or more electrodes comprises a first electrode and a second electrode, wherein the first electrode is disposed on the first surface, and wherein the second electrode is disposed on the second surface.

19. A method for treating tissue, the method comprising:
navigating a medical system through a body lumen to a target tissue, the medical system comprising:
a medical device comprising a shaft, wherein the shaft includes a working channel; and
an end cap coupled to a distal end of the shaft, wherein the end cap includes a protrusion having an electrode at a distal end of the protrusion, and wherein at least a distal portion of the protrusion is flat;
extending a medical instrument through the working channel of the shaft;
grasping a portion of the target tissue with the medical instrument to apply traction to the target tissue;
energizing the electrode; and
manipulating one or more of the shaft of the medical device or the medical instrument such that at least a portion of the electrode contacts a portion of the target tissue to cut the target tissue.

20. The method of claim 19, wherein the protrusion includes a distal edge, and wherein the distal edge is curved.
